(19) United States
(12) Patent Application Publication
(10) Pub. No.: US 2014/0004198 A1
(43) Pub. Date: Jan. 2, 2014

(54) GLP-1 PARTICLES AND COMPOSITIONS

(71) Applicants: Per Balschmidt, Hoersholm (DK); Joern Drustrup, Farum (DK); Kasper Huus, Gentofte (DK)

(72) Inventors: Per Balschmidt, Hoersholm (DK); Joern Drustrup, Farum (DK); Kasper Huus, Gentofte (DK)

(73) Assignee: Novo Nordisk A/S, Bagsvaerd (DK)

(21) Appl. No.: 13/980,166

(22) PCT Filed: Jan. 19, 2012
(86) PCT No.: PCT/EP2012/050785

§ 371 Date: Sep. 20, 2013

Related U.S. Application Data
(60) Provisional application No. 61/436,804, filed on Jan. 27, 2011.

(30) Foreign Application Priority Data
Jan. 19, 2011 (EP) 11151456.8

(51) Int. Cl. A61K 9/14; A61K 38/26

(52) U.S. Cl. CPC A61K 9/14; A61K 38/26
USPC 424/489; 514/11.7

(57) ABSTRACT

The present invention relates to compositions and particles comprising a GLP-1 compound, a divalent metal and a polycationic compound. The invention is characterised that the particle comprises a core and a surrounding layer, the core comprising the GLP-1 compound and the divalent metal, and the surrounding layer comprising the polycationic compound. The invention is particularly useful for the treatment of metabolic diseases, including diabetes.

### TECHNICAL FIELD

The present invention relates to the field of pharmaceutical compositions comprising glucagon-like peptide-1 (GLP-1) compounds and methods of making them.

### BACKGROUND

Glucagon-like peptide-1 (GLP-1) is a gut hormone secreted in the body by the intestinal L cells. The natural active forms of GLP-1 are GLP-1-(7-37) and GLP-1-(7-36)NH2. GLP-1 and its analogs are promising treatments of diabetes mellitus, thanks to their ability to increase insulin secretion from the pancreas, insulin-sensitivity in both alpha cells and beta cells, satiety, and to decrease glucagon secretion from the pancreas.

Like most pharmaceutically relevant proteins and peptides, GLP-1 compounds are poorly absorbed through biological membranes. Therefore, they are typically administered by the parenteral route, by subcutaneous injection. In addition, GLP-1 compounds are unstable due to susceptibility towards various water catalyzed reactions when formulated into an aqueous solution.

The natural GLP-1 has a short half life in the body, few minutes, because it is rapidly degraded by the enzyme dipeptidyl peptidase-4. To overcome this disadvantage, sustained release technologies are the subject of considerable research. One approach is to prepare a suspension of the active ingredient which upon administration is slowly dissolved and released to the blood stream. In this perspective, analogs of the natural GLP-1 are being developed. Liraglutide (Arg³⁴, Lys²⁶(N^{ε}-(γ-Glu(N⁺-hexadecanoyl)))-GLP-1(7-37)) or also named N^{ε26}-[(4S)-4-carboxy-4-(hexadecanoylamino)butanoyl]-[Arg³⁴]-GLP-1-(7-37)-peptide) is one of them. It is commercialized under the trademark Victoza® as a once daily injectable medication. In this formulation, liraglutide has a pharmacokinetic profile (PK) lasting 1 day upon subcutaneous administration. This is a major achievement but there is still a need to lower the frequency of injections for the patients. The development of a once weekly injection medication would be another major achievement.

Some pharmaceutical compositions of the prior art combine GLP-1 compounds with a basic polypeptide and a divalent metal ion, such as zinc (WO02/098348) into particles in order to control the drug release. However, the compositions of the prior art are still not satisfactory and there is still a need for a GLP-1 product with a reduced frequency of injections, with reduced associated side effects and with advantageous physical properties.

### SUMMARY

The invention relates to new GLP-1 compositions.

The invention is based on the recognition that GLP-1 particles with a specific morphology present beneficial properties. Surprisingly, it has been found that the morphology wherein a polycationic compound forms a layer around a core comprising GLP-1 and a divalent metal is associated with a significant increase in the time of action of the GLP-1 compound in the body while maintaining low or minimizing the problems encountered with GLP-1 formulations.

The invention may also solve further problems that will be apparent from the disclosure of the exemplary embodiments.

In one aspect, the invention relates to a particle comprising a GLP-1 compound, a divalent metal and a polycationic compound, wherein the particle comprises a core and a surrounding layer, the core comprising the GLP-1 compound and the divalent metal, and the surrounding layer comprising the polycationic compound.

In another aspect, the invention relates to a pharmaceutical composition comprising particles, the particles comprising a core and a surrounding layer, the core comprising a GLP-1 compound and a divalent metal, and the surrounding layer comprising a polycationic compound.

In another aspect, the invention relates to methods for the preparation of such particles or compositions.

In another aspect, the invention relates to the use of such particles or compositions for the treatment of diabetes.

In one aspect, the invention provides an improved sustained release GLP-1 composition, with an increased duration of action of the GLP-1 compound. Also or alternatively, in another aspect, the invention provides a sustained release GLP-1 composition with improved physical properties, such as physical stability, a smooth injection through fine needles, an easy re-suspension. Also or alternatively, in another aspect, the invention provides a sustained release GLP-1 composition with improved chemical properties, such as a higher concentration of active ingredient available to patients, a higher control of the particles size, a reduced release of free components from the particles. Also or alternatively, in another aspect, the invention provides a sustained release GLP-1 composition with improved side effects, such as a lower burst release, a lower tissue reaction especially at injection site, a lower histamine release.

In another aspect, the invention provides an improved method of making a GLP-1 composition. Also or alternatively, in another aspect, the invention provides a simple method, with no or limited external intervention, e.g. where a final pH adjustment is avoided. Also or alternatively, in another aspect, the invention provides a method applicable for sterile conditions.

In another aspect, the invention provides a treatment with a reduced frequency in injections for patients.

The invention may also solve further problems that will be apparent from the disclosure of the exemplary embodiments.

### BRIEF DESCRIPTION OF DRAWINGS

FIG. 1 shows the zinc-mediated precipitation of liraglutide with various values of zinc:liraglutide molar ratio and various pH.

FIG. 2 shows the degree of protamine binding to precipitated zinc-liraglutide complex with various molar ratio of protamine per liraglutide.

FIG. 3 shows the degree of protamine binding to precipitated zinc-liraglutide complex with various pH values.

### DESCRIPTION

The invention relates to novel GLP-1 particles and compositions. The novel particles and compositions of the invention can be used for the treatment of diabetes, such as type 2 diabetes. The particles and compositions are useful as a treatment with a frequency of administration below once per day.

The particles and compositions of the invention give a suitable sustained release PK profile upon subcutaneous administration, are of appropriate and controlled size, are easily resuspendable upon storage, and are injectable through fine injection needles. They also allow the GLP-1 compound to be formulated at high concentrations, allowing a longer time of action. This specific morphology of the particle also reduces undesired side effects.

The features of the invention will be better understood in the description that follows.

In one aspect, the invention relates to a particle comprising a GLP-1 compound, a divalent metal and a polycationic compound, wherein the particle comprises a core and a surrounding layer, the core comprising the GLP-1 compound and the divalent metal, and the surrounding layer comprising the polycationic compound.

The term “particle” as used herein means a solid material complex, with a GLP-1 and divalent metal core, and with a polycationic compound present on the surface of the core.

Non-limiting examples of GLP-1 compound include a natural GLP-1, a GLP-1 analogue, or a GLP-1 derivative.

In its broadest sense, the term “natural GLP-1” refers to a naturally occurring molecule of the glucagon family of peptides or of the family of exendins. The glucagon family of peptides are encoded by the pre-proglucagon gene and encompasses three small peptides with a high degree of homology, i.e. glucagon (1-29), GLP-1 (1-37) and GLP-2 (1-33). The term “natural GLP-1” also refers to the human GLP-1(7-37), the sequence of which is disclosed as SEQ ID NO:1 in WO 2006097537 and included herein by reference, and to the human GLP-1(7-36)NH2. Exendins are peptides expressed in lizards and like GLP-1, are insulinotropic. Examples of naturally occurring exendins are exendin-3 and exendin-4.

In a particular embodiment, the term “natural GLP-1” refers to glucagon (1-29), GLP-1 (1-37) and GLP-2 (1-33), the human GLP-1(7-37)), the human GLP-1(7-36)NH2, exendin-3 and exendin-4.

In a particular embodiment, the term “GLP-1 compound” does not include the human GLP-1(7-36)NH2. In a particular embodiment, the term “GLP-1 compound” does not include the human GLP-1(7-37).

In a particular embodiment, the term “GLP-1 compound” does not include glucagon.

In a particular embodiment, the term “GLP-1 compound” does not include the human GLP-1(7-36)NH2 and glucagon or does not include human GLP-1(7-36)NH2, human GLP-1(7-37) and glucagon.

In a more particular embodiment, the term “natural GLP-1” only refers to the human GLP-1(7-37).

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been added to the peptide. Such addition or deletion of amino acid residues can take place at the N-terminal of the peptide and/or at the C-terminal of the peptide.

In its broadest sense, the term “GLP-1 analogue” or “analogue of GLP-1” as used herein refers to an analogue of a natural GLP-1. It does not include a natural GLP-1 as such as defined herein. In particular, the term “GLP-1 analogue” does not include glucagon (1-29), GLP-1 (1-37) and GLP-2 (1-33), the human GLP-1(7-37)), the human GLP-1(7-36)NH2, exendin-3 and exendin-4.

In a particular embodiment, the term “GLP-1 analogue” or “analogue of GLP-1” as used herein refers to an analogue of human GLP-1(7-37) or GLP-1(7-36)NH2.

Non-limiting examples of GLP-1 analogues comprise exenatide and taspoglutide.

In a particular embodiment, the “GLP-1 analogues” comprise analogues with a maximum of 17 amino acid modifications (i.e. up to 17 amino acids have been modified in total, where the changes can be amino acid substitutions, additions and/or deletions) compared to a natural GLP-1 of reference or, in particular, compared to human GLP-1-(7-36)NH2 or GLP-1(7-37).

All amino acids for which the optical isomer is not stated is to be understood to mean the L-isomer.

In embodiments of the invention a maximum of 17 amino acids have been modified (substituted, deleted, added or any combination thereof) relative to a natural GLP-1 of reference or, in particular, relative to human GLP-1-(7-36)NH2 or GLP-1(7-37). In embodiments of the invention a maximum of 15 amino acids have been modified. In embodiments of the invention a maximum of 10 amino acids have been modified. In embodiments of the invention a maximum of 8 amino acids have been modified. In embodiments of the invention a maximum of 7 amino acids have been modified. In embodiments of the invention a maximum of 6 amino acids have been modified. In embodiments of the invention a maximum of 5 amino acids have been modified. In embodiments of the invention a maximum of 4 amino acids have been modified. In embodiments of the invention a maximum of 3 amino acids have been modified. In embodiments of the invention a maximum of 2 amino acids have been modified. In embodiments of the invention 1 amino acid has been modified relative to relative to a natural GLP-1 of reference or, in particular, relative to human GLP-1-(7-36)NH2 or GLP-1(7-37). In a particular embodiment, the amino acid modifications of this paragraph are relative to human GLP-1(7-37).

In a particular embodiment, the GLP-1 analogues comprise a substitution of the amino acid residue in position 34 from Lys to Arg, i.e. Arg³⁴, compared to GLP-1(7-37) or GLP-1-(7-36)NH2. In a particular embodiment, the GLP-1 analogues have a substitution of the amino acid residue in position 8 from Ala to Aib (alpha-amino-iso-butyric acid), i.e. Aib⁸. In a particular embodiment, the GLP-1 analogues have the Arg³⁴ substitution, the Aib⁸ substitution, or both the Arg³⁴ and Aib⁸ substitutions, and possibly one more amino acid modification compared to GLP-1(7-37) or GLP-1-(7-36)NH2. In a particular embodiment, the amino acid modifications of this paragraph are relative to human GLP-1(7-37).

The term “derivative” as used herein in relation to a peptide means a chemically modified peptide or an analogue thereof, wherein at least one substituent has been attached to the unmodified peptide or an analogue thereof, i.e. a peptide which has been covalently modified. The substituent may also be referred to as a “side chain”. The peptide to which the substituent(s) is attached may also be referred to as the “parent” peptide.

In its broadest sense, the term “GLP-1 derivative” or “derivative of GLP-1” as used herein refers to a derivative of a parent peptide selected from a natural GLP-1 or an analogue thereof. It does not include a natural GLP-1 as such as defined herein. In particular, the term “GLP-1 derivative” does not include glucagon (1-29), GLP-1 (1-37) and GLP-2 (1-33), the human GLP-1(7-37)), the human GLP-1(7-36)NH2, exendin-3 and exendin-4.

In a particular embodiment, the term “GLP-1 derivative” or “derivative of GLP-1” refers to a derivative of a parent peptide selected from human GLP-1(7-37) or GLP-1(7-36)NH2 or an analogue thereof.

In a particular embodiment, the term “GLP-1 derivative” or “derivative of GLP-1” as used herein refers to a derivative of a parent peptide selected from a GLP-1 analogue, where said analogue comprises a maximum of 17 amino acid modifications compared to a natural GLP-1 of reference or, in particular, compared to human GLP-1-(7-36)NH2 or GLP-1(7-37), or, in particular, compared to human GLP-1(7-37). In one embodiment, the “GLP-1 derivative”, in particular when defined in comparison to GLP-1(7-37), does not include GLP-1(7-36)NH2.

Typical modifications are amides, carbohydrates, alkyl groups, acyl groups, esters, polyethylene glycol (PEG) groups, sialylation groups, glycosylation groups and the like of a parent peptide. In one embodiment, the parent peptide is a GLP-1 analogue as defined above.

In particular embodiments, the side chain has at least 10 carbon atoms, or at least 15, 20, 25, 30, 35, or at least 40 carbon atoms. In further particular embodiments, the side chain may further include at least 5 hetero atoms, in particular O and N, for example at least 7, 9, 10, 12, 15, 17, or at least 20 hetero atoms, such as at least 1, 2, or 3 N-atoms, and/or at least 3, 6, 9, 12, or 15 O-atoms.

In one embodiment, the term “GLP-1 derivative” refers to acylated GLP-1 parent peptide. In a particular embodiment, the term “GLP-1 derivative” refers to acylated GLP-1 parent peptide where the parent peptide is selected from a GLP-1 analogue comprising a maximum of 17 amino acid modifications compared to a natural GLP-1 of reference or, in particular, compared to human GLP-1-(7-36)NH2 or GLP-1(7-37).

The side chain may be covalently attached to a lysine residue of the GLP-1 parent peptide by acylation. Additional or alternative conjugation chemistry includes alkylation, ester formation, or amide formation, or coupling to a cysteine residue, such as by maleimide or haloacetamide (such as bromo-/fluoro-/iodo-) coupling.

For the preparation, an active ester of the side chain is covalently linked to an amino group of a lysine residue, preferably the epsilon amino group thereof, under formation of an amide bond (this process being referred to as acylation).

Preferred side chains include, for example, fatty acids and fatty diacids. The term fatty acid refers to aliphatic monocarboxylic acids having from 4 to 28 carbon atoms. The fatty acid may be branched or unbranched. The fatty acid is preferably even numbered. The fatty acid may be saturated or unsaturated. The term fatty diacid refers to fatty acids as defined above but with an additional carboxylic acid group in the omega position. Thus, fatty diacids are dicarboxylic acids.

In a particular embodiment, the side chain(s) is a fatty acid having 10 to 20 carbon atoms, and preferably 14 to 20 or 16 to 18 carbon atoms, optionally with a spacer.

In a particular embodiment, the side chain(s) is a fatty acid of formula Chem.1: HOOC(CH₂)ₘCO, wherein m is an integer from 8 to 18, optionally with a linker. In a particular embodiment, m is an integer from 12 to 18 or from 14 to 16.

In a particular embodiment, the side chain(s) is selected from the group consisting of HOOC(CH₂)₁₄CO—, HOOC(CH₂)₁₆CO—, HOOC(CH₂)₂₂CO—, CH₃(CH₂)₁₄CO—, CH₃(CH₂)₁₆CO— and CH₃(CH₂)₁₈CO—.

In one embodiment, the term “GLP-1 derivative” comprises or refers to a monoacylated GLP-1 parent peptide, i.e. a GLP-1 parent peptide comprising only one acylation as defined above.

In a particular embodiment, the side chain is a fatty acid or a fatty diacid of which an acid group forms an amide bond with the epsilon amino group of a lysine residue in the GLP-1 compound, preferably via a spacer. In one embodiment, said lysine residue is Lys²⁶, especially when the parent peptide is human GLP-1(7-37), GLP-1(7-36)NH2 or a GLP-1 analogue.

In a particular embodiment, the side chain is attached to the parent peptide by means of a linker. In a particular embodiment, the linker comprises a γ-glutamic acid (γ-Glu) and/or 1, 2 or 3 OEG molecules. In γGlu the gamma carboxy group of the amino acid glutamic acid is used for connection to another linker element, or to the epsilon-amino group of lysine. An OEG molecule is also named a di-radical of 8-amino-3,6-dioxaoctanic acid, and/or it may be represented by the formula Chem. 2: —NH—(CH2)2-O—(CH2)2-O—CH2-CO—.

The linker may include one or more γGlu, and/or one or more OEG. More in particular, the γGlu and OEG linker elements may, independently, be used p times where p is zero or an integer in the range of 1-3. Examples of preferred linkers are γGlu, γGlu-2xOEG, and γGlu-3xOEG where in all cases the alpha-amino group of Glu forms an amide bond with the carboxy group of the protracting moiety.

In a particular embodiment, the GLP-1 derivative is a derivative of a GLP-1 analogue which comprises the Arg³⁴ substitution or the Arg³⁴ and the Aib⁸ substitutions compared to human GLP-1(7-37), GLP-1(7-36)NH2 and which comprises a side chain attached to Lys²⁶. In a particular embodiment said side chain is a fatty acid as defined above, especially a fatty acid of formula Chem.1, with m being an integer from 8 to 18, optionally with a linker being γGlu.

In one embodiment, the GLP-1 derivative is as defined in the patent applications WO 98/08871 and WO 06/097537, entirely included herein by reference. Non-limiting examples of monoacylated GLP-1 derivatives can be found in those applications.

Non-limiting examples of GLP-1 derivatives also include:
N^{ε37}-[2-[2-[2-[[2-[2-[2-[[(4S)-4-carboxy-4-[[4-[(19-carboxynonadecanoylamino)methyl]cyclo hexanecarbonyl]amino]butanoyl]amino]ethoxy]ethoxy]acetyl]amino]ethoxy]ethoxy]ace tyl[Imp⁷,Glu²²,Arg²⁸,Arg³⁴,Lys³⁷]-GLP-1-(7-37)-peptide;
   N^{ε26}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Aib⁸,Arg³⁴]-GLP-1-(7-37)-peptide, also called semaglutide;
N^{ε26}-[(4S)-4-carboxy-4-(hexadecanoylamino)butanoyl]-[Arg³⁴]-GLP-1-(7-37)-peptide, also called liraglutide;
N^{ε26}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl],N^{ε37}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide;
N^{ε26}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl],N^{ε37}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide;
lixisenatide;
albiglutide;
dulaglutide.

In a particular embodiment, the GLP-1 derivative is liraglutide or is semaglutide.

In one embodiment, the term “GLP-1 derivative” comprises or refers to alkylated GLP-1 compounds.

The chemically modified derivatives of natural GLP-1 can be prepared for example as described in U.S. Pat. No. 6,451,762 or in Knudsen et. al. (2000) J Med Chem 43, 1664-1669.

Non-limiting examples of divalent metal include zinc (Zn), calcium (Ca), manganese (Mn) or magnesium (Mg). As non-limiting examples, the source of zinc may be zinc chloride, zinc acetate, zinc sulphate or zinc oxide. Amongst these, at least, zinc acetate allows an easy preparation of solutions.

Non-limiting examples of polycationic compound include protamine, chitosan, a chitosan derivative, polylysine or polyarginine. As non-limiting examples, the polycationic compound is protamine and comes from protamine chloride, protamine acetate, protamine sulphate.

The particle of the invention comprises a core.

In one embodiment, the core of the particle of the invention comprises a GLP-1 compound and a divalent metal. In another embodiment, the core of the particle of the invention consists of a GLP-1 compound and a divalent metal.

In the core, the GLP-1 and the metal molecules are co-precipitated and form a homogenous mixture. The term “homogeneous mixture” as used herein means that each component present in the core is evenly distributed. In one embodiment, a solution of the divalent metal is added to a solution of GLP-1 compound leading to precipitation of GLP-1/metal particles. In another embodiment, a solution of the GLP-1 compound is added to a solution of the divalent metal leading to precipitation of GLP-1/metal particles.

In one embodiment, the GLP-1 and metal mixture forms an amorphous complex. The term “amorphous” as used herein means that the molecules are organised randomly and are not present in a crystalline state. In another embodiment, the GLP-1 and metal mixture forms a crystalline complex, or comprises both amorphous and crystalline complexes. The term “crystalline” as used herein means that the molecules are ordered into one or several definable forms or structures as determined by polarized light microscopy.

In another embodiment, the core of the particle comprises no or substantially no polycationic compound, no or substantially no polycationic peptide, no or substantially no protamine, no or substantially no chitosan, no or substantially no polylysine and/or no or substantially no polyarginine.

The absence of polycationic compound, such as protamine, within the core helps preventing the formation of a gel consistency with the GLP-1 compound and allows an improved control of the size of the particles. Indeed, it has been found that the mixing of a GLP-1 compound with a polycationic compound, such as liraglutide and protamine in solutions, leads to the formation of mixture with a gel texture, which impacts the particle size distribution.

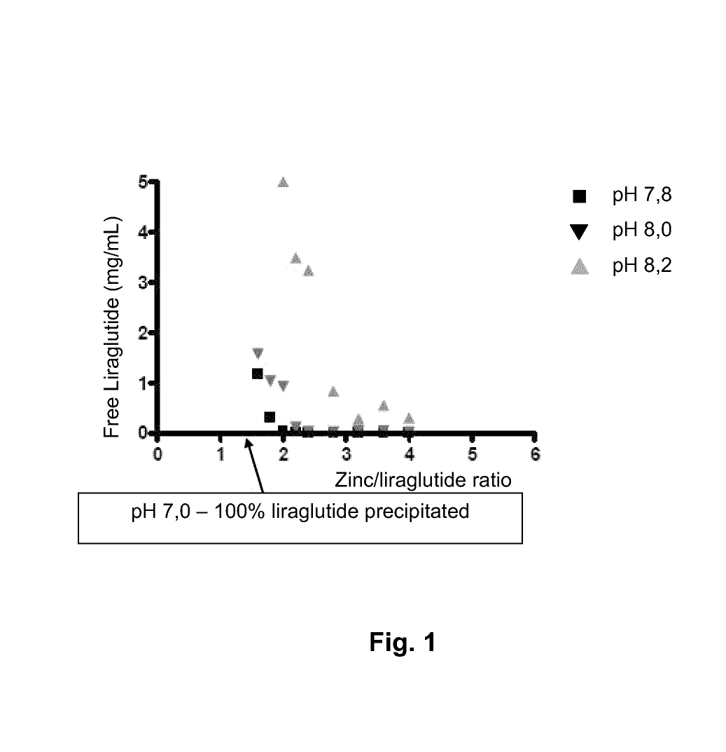

In the core of the particle, the divalent metal stabilizes the particle and reduces the particle solubility. The divalent metal precipitates the GLP-1 compound. This reduces the release of free GLP-1 out of the particles into the supernatant, i.e. into the composition comprising the particles, before it is injected. (See FIG. 1 and example 1)

In one embodiment, the GLP-1:divalent metal molar ratio in the core of the particle is 1:≧2 or 1:>2. This means that the core of the particle comprises at least 2 divalent metal molecules per GLP-1 molecule (1:≧2) or more than 2 divalent metal molecules per GLP-1 molecule (1:>2). In another embodiment, the GLP-1:divalent metal molar ratio in the core of the particle is 1:≧2.1 or of 1:>2, 1 or 1:2.1. In another embodiment, the GLP-1:divalent metal molar ratio in the core of the particle is 1:≧2.2 or of 1:>2, 2 or 1:2.2. In another embodiment, the GLP-1:divalent metal molar ratio in the core of the particle is between 1:2.0 and 1:5, between 1:2.1 and 1:5, between 1:2.0 and 1:4, between 1:2.1 and 1:4, between 1:2.0 and 1:3, between 1:2.1 and 1:3, between 1:2.0 and 1:2.4, between 1:2.1 and 1:2.4 or between 1:2.1 and 1:2.3. These embodiments avoid excess of divalent metal molecules and limit the presence of free divalent metal molecules in the supernatant which may otherwise generate unwanted tissue reaction. These embodiments advantageously also limit the presence of free GLP-1 in the supernatant.

The above ratios are associated with a reduction or with a total prevention (isophane ratios) of undesired release of free GLP-1 into the composition comprising the particles. This strategy to avoid as much GLP-1 as possible in the supernatant minimizes the burst release and related side effects such as injection site reaction. It also increases the chemical and physical stability of the particles and of the GLP-1 molecule itself. It also helps controlling and increasing the sustained-release of the GLP-1 compound after injection into the body and the associated protraction action.

The particle of the invention also comprises a layer surrounding the core. This layer surrounds and covers the core of the particle. In other words, the layer coats the surface of the core and is a part of the particles. The polycationic compound forming the surrounding layer is attached onto the surface of the core of the particle. This contributes to limit the presence of free polycationic compound in the surpernatant. In particular, the term “layer” does not designate a composition in which the core would simply be suspended.

In one embodiment, this layer covers the major part of the core, so that only a minor part of the core is in direct contact with the external environment, such as the composition comprising the particles. In one embodiment, this layer covers the entire outer surface of the core, so that no component of the core is in direct contact with the external environment, such as the composition comprising the particles. In one embodiment, this surrounding layer is the outer layer of the particle. It avoids significant concentrations of both free divalent metal and free polycationic compound otherwise found in the supernatant of suspension comprising the particles and associated with a vast histological response at injection site.

The surrounding layer comprises or consists of the polycationic compound, as described above. In one embodiment, the GLP-1:polycationic compound molar ratio in the particle is 1:>0.01. In another embodiment, the GLP-1:polycationic compound molar ratio is 1:0.01-1; 1:>0.1; 1:>0.11; 1:≦0.11; 1:≦0.12; 1:>0.12; 1:0.12-1.0, 15; 1:≦0.13; 1:>0.13; 1:0.13-0.15; 1:0.13; 1:0.14-1:0.15; 1:0.14 or 1:0.15. These embodiments advantageously limit the presence of free polycationic compound in the supernatant. In one embodiment, the particles of the invention have a volumetric diameter below 200 μm. The term “diameter” as used herein designates the diameter of an entire particle. In the context of a composition comprising the particles of the invention, the “diameter” designates the mean diameter of either all or a proportion of the particles. In one embodiment, at least, 50% of the particles of the invention have a volumetric diameter less than 60 μm. In one embodiment, 50% of the particles of the invention have a volumetric diameter less than 40 μm. In one embodiment, 50% of the particles of the invention have a volumetric diameter in the range of 5-35 μm. The particle size distributions including the volumetric diameters can be determined using a Helos particle analyser from Sympatec, that uses a laser diffraction sensor.

In one embodiment, the particles of the invention are comprised in a pharmaceutical composition. In one embodiment, the composition is a suspension of said particles into an aqueous vehicle.

As a further advantage, the GLP-1 compounds are stabilized against chemical and physical degradation when incorporated in a composition.

In one embodiment, the pharmaceutical composition of the invention is a non-aqueous suspension of particles as described above. Therefore, the particles can be suspended in a non-aqueous medium, for example an oil, such as MCT (medium chain triglyceride). The particles and the non-aqueous medium can either be pre-mixed and thereby ready to use, or separately stored. In the latter case, mixing of the particles and the non-aqueous medium has to take place before use.

In another embodiment, the particles can be further incorporated into at least one biodegradable polymer, such as PLGA (poly(lactic-co-glycolic acid), the final combination either present as spheres or rods. The spheres, consisting of both the particles and at least one biodegradable polymer, can either be premixed into an oil such as MCT, and thereby ready to use, or separately stored. In the latter case, mixing of the spheres and medium has to take place before use. In addition, the obtained spheres can be stored separately from an aqueous medium. In this case, mixing of the spheres and aqueous medium has to take place shortly before use, in order to avoid the biodegradable polymer to degrade prior to dosing.

When the pharmaceutical composition is a non-aqueous suspension, the particles are preferably obtained by isolation and drying from an aqueous suspension, or made by appropriate spray drying.

In another embodiment, the pharmaceutical composition of the invention is an aqueous suspension of particles as described above. Therefore, the particles are present in an aqueous suspension which may also comprise one or several of the followings:

a tonifier or isotonic agent, such as sodium chloride, glycerol, propylene glycol, mannitol, sucrose, trehalose;

a buffer, such as TRIS (tris(hydroxymethyl)aminomethane), HEPES (4-(2-hydroxyethyl)-1-piperazineethanesulfonic acid), GlyGly etc, possibly with a pH adjusting agent such as hydrochloric acid, sodium hydroxide, acetic acid etc;

a preservative agent, such as phenol, m-cresol, benzyl alcohol etc, and mixtures thereof;

an additional stabilizer, such as amino acids, surfactants etc.

When the pharmaceutical composition is an aqueous suspension, the particles are preferably used as such and further processed in the aqueous media or made by appropriate spray drying, followed by a re-suspension into a new aqueous media.

The invention is particularly useful as an injectable, for example, without limitation, for subcutaneous, intramuscular or intraperitoneal administration route.

In one embodiment, the particles, when dissolved or suspended in water, or the composition comprising the particles of the invention, have a pH between 4 and 8.2. In another embodiment, said pH is between 7.2 and 8.2, between 7.4 and 8.2, between 7.4 and 7.9, between 7.6 and 8.0 or between 7.7 and 7.9, or said pH is 7.4, 7.6, 7.8, 8.0 or 8.2. Thus, it is possible to achieve a suspension with a narrow particle size, leaving no GLP-1 dissolved in the supernatant of the suspension. If not specified otherwise, a pH value is considered at around room temperature, e.g. 20-26° C. or 23-25° C.

In one embodiment, the particles or the compositions of the invention allow a time release of the GLP-1 compound after injection into the body (in vivo plasma profile) of more than 24 hours, of more than 72 hours, of up to 3 days, 4 days, 5 days, 7 days or of up to 8 days.

In another embodiment, the particles or the compositions of the invention of the invention allow a time release of the GLP-1 compound after injection into the body (in vivo plasma profile) of more than 7 days, more than 8 days, more than 9 days, more than 10 days, more than 14 days, more than 15 days, more than 20 days, more than 21 days, more than 29 days, more than 30 days, more than 31 days, or of up to 1 month.

In particular, when combining the particles with either a non-aqueous medium or a biodegradable polymer, or both can provide an even further sustained release profile for the GLP-1 molecule, as compared to the release profile of GLP-1 from particles merely suspended in an aqueous medium.

In one embodiment, the composition comprising the particles of the invention, especially a suspension of the particles, comprise a GLP-1 compound concentration of up to 100 mg/mL or between 0.1 and 100 mg/mL. In one embodiment, the composition comprises a GLP-1 compound concentration between 35 and 45 mg/mL, between 37 and 43 mg/mL or of 40 mg/mL. For example, pharmaceutical compositions of particles comprising up to 40 mg/mL of liraglutide have been obtained.

In one embodiment, the particles of the invention present low burst release when injected into a target body.

In one embodiment, the particles of the invention, when in the form of a suspension, present a low sedimentation rate. For example, the particles of the invention can have a sedimentation percentage higher than 80% at 5 minutes after being resuspended, such as 90 or 95%. A sedimentation percentage higher than 80% at 5 minutes means that the solids in this suspension settle less than 20% (of the total height of the suspension) within these 5 minutes.

In one embodiment, the particles of the invention allow a pharmaceutical composition comprising them to be readily injectable through an injection needle finer than or equal to a 28 G (gauge) or a 30 G regular walled needle, for example with a dose force, i.e. injection force, lower than 25N (newtons) at a dosing rate of at least 50 μL/sec.

In another embodiment, the Zn:GLP-1 molar ratio does not exceed a value where zinc is present in large amounts in the supernatant at a sufficiently high pH value in order to avoid the formation of zinc hydroxide (Zn(OH)₂) precipitates. These Zn(OH)₂ precipitates may appear for example when the zinc and GLP-1 compounds are in the form of an amorphous complex in the core, if made at a too high pH value. These zinc hydroxide precipitates may cause serious tissue reaction at the injection site.

By isophane titration studies, it has been possible to determine the required minimum concentration of divalent metal, as well as of polycationic compound, in order to minimize the amount of all three components in the supernatant at the investigated pH.

As shown in FIG. 1, the pH of the composition influences the particle solubility. For example, at room temperature and pH 7, a minimum of 1.3 zinc per GLP-1 molecule is needed to have no free GLP-1 released into the supernatant, and at room temperature and pH 7.8, a minimum of 2 zinc per GLP-1 molecule is needed to completely prevent the release of free GLP-1 into the supernatant. A Zn:GLP-1 molar ratio between 2.0:1 and 2.2:1, or of 2.0:1, 2.1:1 or 2.2:1, is used to compensate the pH deviation that may occur in a pharmaceutical formulation, ensuring that no free GLP-1 in released into the supernatant at any time during the formulation storage.

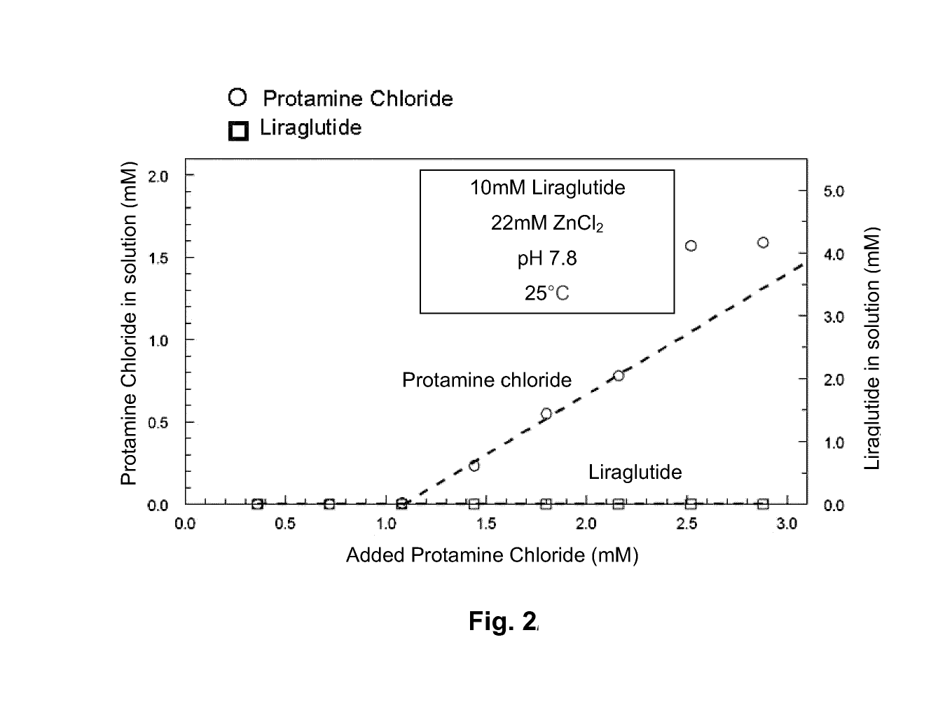

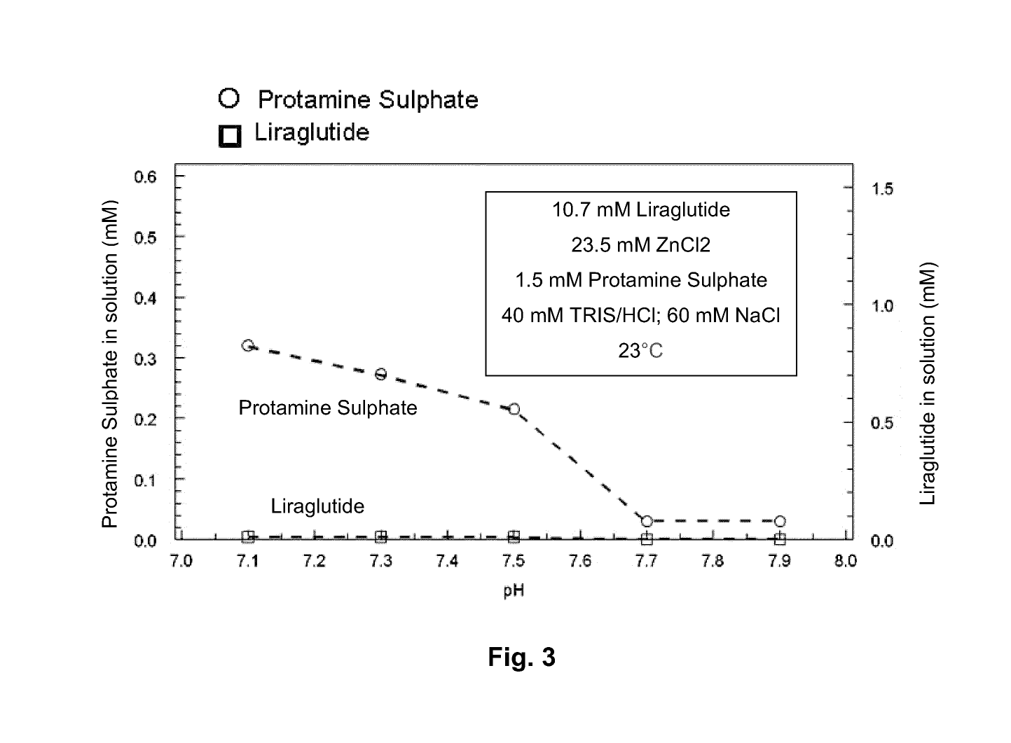

As shown in FIG. 2, the molar ratio between the polycationic compound and the GLP-1 compound influences the amount of free polycationic compound released from the particle into the supernatant. As shown in FIG. 3, the pH also impacts on the release of free polycationic compound. For example, at pH 7.8, with 0.11 molecule of protamine per molecule of liraglutide in the core, there is no free protamine in the supernatant. At a molar ratio of 0.13, 0.14 or 0.15 polycationic compound per 1 GLP-1 compound it is possible to minimize the protamine in the supernatant in the pH range of 7.7-7.9.

Therefore, the following embodiments are also part of the present invention: Particles comprising a GLP-1 compound, a divalent metal and a polycationic compound, wherein said particles comprise a core and a surrounding layer, the core comprising the GLP-1 compound and the divalent metal, and the surrounding layer comprising the polycationic compound, wherein:

1—the GLP-1:divalent metal molar ratio is 1:>2.
2—the GLP-1 compound is selected from the group consisting of GLP-1 analogues and GLP-1 derivatives and the GLP-1:divalent metal molar ratio is 1:>2.
3—the GLP-1 compound is selected from the group consisting of GLP-1 analogues and GLP-1 derivatives and the GLP-1:divalent metal molar ratio is 1:2.1-2.4.
4—the GLP-1 compound is selected from the group consisting of GLP-1 analogues and GLP-1 derivatives and the particles are comprised in a pharmaceutical composition having a pH between 7.4 and 8.0 or 7.7 and 8.0.
5—the GLP-1 compound is selected from the group consisting of GLP-1 analogues and GLP-1 derivatives, the GLP-1:divalent metal molar ratio is 1:2.1-2.4 and the particles are comprised in a pharmaceutical composition having a pH between 7.4 and 8.0 or between 7.4 and 7.9.
6—the GLP-1 compound is selected from the group consisting of GLP-1 analogues and GLP-1 derivatives, the GLP-1:divalent metal molar ratio is 1:2.1-2.4 and the particles are comprised in a pharmaceutical composition having a pH between 7.7 and 8.0 or between 7.7 and 7.9.
7—the GLP-1:divalent metal molar ratio is 1:>2 and the GLP-1:polycationic compound molar ratio is 1:0.01-1.
8—the GLP-1:divalent metal molar ratio is 1:2.1-2.4 and the GLP-1:polycationic compound molar ratio is 1:0.13-0.15.
9—the GLP-1 compound is selected from the group consisting of GLP-1 analogues and GLP-1 derivatives, the GLP-1:divalent metal molar ratio is 1:2.1-2.4 and the GLP-1:polycationic compound molar ratio is 1:0.13-0.15.
10—the GLP-1 compound is selected from the group consisting of GLP-1 analogues and GLP-1 derivatives, the GLP-1:divalent metal molar ratio is 1:2.1-2.4, the GLP-1:polycationic compound molar ratio is 1:0.13-0.15 and the particles are comprised in a pharmaceutical composition with a GLP-1 concentration between 35 and 45 mg/ml or is 40 mg/ml of the composition.
11—the GLP-1 compound is selected from the group consisting of GLP-1 analogues and GLP-1 derivatives, the GLP-1:polycationic compound molar ratio is 1:0.13-0.15, and the particles are comprised in a pharmaceutical composition having a pH between 7.4 and 8.0 or between 7.7 and 8.0.
12—the GLP-1 compound is selected from the group consisting of GLP-1 analogues and GLP-1 derivatives, the GLP-1:divalent metal molar ratio is 1:2.1-2.4, the GLP-1:polycationic compound molar ratio is 1:0.13-0.15 and the particles are comprised in a pharmaceutical composition having a pH between 7.4 and 8.0 or between 7.4 and 7.9.
13—the GLP-1 compound is selected from the group consisting of GLP-1 analogues and GLP-1 derivatives, the GLP-1:divalent metal molar ratio is 1:2.1-2.4, the GLP-1:polycationic compound molar ratio is 1:0.13-0.15, the particles are comprised in a pharmaceutical composition having a pH between 7.4 and 8.0 or between 7.4 and 7.9 and the GLP-1 concentration is between 35 and 45 mg/ml or is 40 mg/ml in the composition.
14—the GLP-1 compound is selected from the group consisting of GLP-1 analogues and GLP-1 derivatives, the GLP-1:divalent metal molar ratio is 1:2.1-2.4, the GLP-1:polycationic compound molar ratio is 1:0.13-0.15 and the particles are comprised in a pharmaceutical composition having a pH between 7.7 and 8.0 or between 7.7 and 7.9.
15—the GLP-1 compound does not include human GLP-1(7-36)NH2 and glucagon or does not include human GLP-1(7-36)NH2, human GLP-1(7-37) and glucagon.
16—the GLP-1 compound is a GLP-1 analogue or a GLP-1 derivative wherein the GLP-1 analogue is selected from the group consisting of analogues of human GLP-1(7-37) or GLP-1(7-36)NH2 with a maximum of 17 amino acid modifications compared to human GLP-1-(7-36)NH2 or GLP-1(7-37) and the GLP-1 derivative is selected from the group consisting of a derivative of human GLP-1(7-37), of GLP-1(7-36)NH2 or of an analogue thereof with a maximum of 17 amino acid modifications.
17—the GLP-1 compound is a GLP-1 derivative.
18—the GLP-1 compound is a GLP-1 derivative of human GLP-1(7-37), of GLP-1(7-36)NH2 or of an analogue thereof with a maximum of 17 amino acid modifications.
19—the GLP-1 compound is a GLP-1 derivative selected from the group consisting of amidated parent peptide, alkylated parent peptide, acylated parent peptide, esterified parent peptide, PEGylated parent peptide and/or sialylated parent peptide and the parent peptide is human GLP-1(7-37), GLP-1(7-36)NH2 or an analogue thereof with a maximum of 17 amino acid modifications.
20—the GLP-1 compound is a GLP-1 derivative selected from the group consisting of acylated GLP-1 parent peptide, the parent peptide is human GLP-1(7-37), GLP-1(7-36)NH2 or an analogue thereof with a maximum of 17 amino acid modifications, the parent peptide is acylated with a lipophilic substituent selected from the group consisting of aliphatic monocarboxylic or dicarboxylic acids having from 4 to 28 carbon atoms.
21—the GLP-1 compound is a GLP-1 derivative selected from the group consisting of acylated GLP-1 parent peptide, the parent peptide is human GLP-1(7-37), GLP-1(7-36)NH2 or an analogue thereof with a maximum of 17 amino acid modifications, the parent peptide is acylated with a lipophilic substituent selected from the group consisting of aliphatic monocarboxylic or dicarboxylic acids having from 14 to 20 carbon atoms.
22—the GLP-1 compound is selected from the group consisting of liraglutide, semaglutide, taspoglutide, exenatide, lixisenatide, albiglutide, dulaglutide, or
N^{ε37}-[2-[2-[2-[[2-[2-[2-[[(4S)-4-carboxy-4-[[4-[(19-carboxynonadecanoylamino)methyl]cyclo hexanecarbonyl]amino]butanoyl]amino]ethoxy]ethoxy]acetyl]amino]ethoxy]ethoxy]acetyl]-[Imp⁷,Glu²²,Arg²⁶,Arg³⁴,Lys³⁷]-GLP-1-(7-37)-peptide or
N^{ε26}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl], N^{ε37}-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide or N^{ε26}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl], N^{ε37}-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide.
   23—any of embodiments 1 to 22 wherein the divalent metal is zinc and the polycationic compound is protamine.
   24—any of embodiments 1 to 22 wherein the particle size is less than 200 micrometers in diameter.
   25—any of embodiments 1 to 22 wherein the divalent metal is zinc, the polycationic compound is protamine and the particle size is less than 200 micrometers in diameter.
   26—embodiment 24 or embodiment 25 wherein at least 50% of the particles have a volumetric diameter less than 60 μm, or 50% or at least 50% of the particles have a volumetric diameter less than 40 μm or 50% or at least 50% of the particles have a volumetric diameter in the range of 5-35 μm.
   For clarity reasons, the specific combinations of any one of embodiments 1 to 14 with any one of embodiments 15 to 26 are not reported here in writing but should be considered to be part of the disclosure. The same applies to the specific combination of embodiment 23 with any one of embodiments 1 to 14 and with any one of embodiments 15 to 22; to the specific combination of embodiment 24 with any one of embodiments 1 to 14 and with any one of embodiments 15 to 22; to the specific combination of embodiment 25 with any one of embodiments 1 to 14 and with any one of embodiments 15 to 22; and to the specific combination of embodiment 26 with any one of embodiments 1 to 14 and with any one of embodiments 15 to 22.
   27—the GLP-1 compound is liraglutide, the divalent metal is zinc, and the liraglutide:zinc molar ratio is 1:2.0-2, 4 or 1:2.1-2.4
   28—the GLP-1 compound is liraglutide, the divalent metal is zinc, and the liraglutide:zinc molar ratio is 1:>2 and the particles are comprised in a pharmaceutical composition, the liraglutide concentration being between 35 and 45 mg/ml or is 40 mg/ml in the composition;
   29—the GLP-1 compound is liraglutide, the divalent metal is zinc, and the liraglutide:zinc molar ratio is 1:2.1-2.4 and the particles are comprised in a pharmaceutical composition, the liraglutide concentration being between 35 and 45 mg/ml or is 40 mg/ml in the composition;
   30—the GLP-1 compound is liraglutide, the divalent metal is zinc, the liraglutide:zinc molar ratio is 1:2.0-2, 4:1, 1:2.1-2.4 or is 1:2.2, in a composition having a pH≧7.7, a pH≧7.8, a pH between 7.7 and 8.0, a pH between 7.7 and 7.9, a pH of 7.7, a pH of 7.8 or a pH of 7.9;
   31—the GLP-1 compound is liraglutide, the polycationic compound is protamine, and the liraglutide:protamine molar ratio is 1:0.13-0.15.
   32—the GLP-1 compound is liraglutide, the polycationic compound is protamine, and the liraglutide:protamine molar ratio is 1:0.13-0.15, in a composition having a pH≧7.7, a pH≧7.8, a pH between 7.7 and 8.0, a pH between 7.7 and 7.9, a pH of 7.7, a pH of 7.8 or a pH of 7.9;
   33—the GLP-1 compound is liraglutide, the polycationic compound is protamine, the liraglutide:protamine molar ratio is 1:0.11 or 1:>0.11 or is 1:≧0.11, in a composition having a pH of 7.8 or a pH 7.8;
   34—the GLP-1 compound is liraglutide, the divalent metal is zinc, the liraglutide:zinc molar ratio is 1:2.0-2, 4 or 1:2.1-2.4 and the cores of the particles comprise no polycationic compound;
   35—the GLP-1 compound is liraglutide, the divalent metal is zinc, the liraglutide:zinc molar ratio is 1:2.0-2, 4:1, 1:2.1-2.4 or is 1:2.2, the cores of the particles comprise no polycationic compound, in a composition having a pH≧7.7, a pH≧7.8, a pH between 7.7 and 8.0, a pH between 7.7 and 7.9, a pH of 7.7, a pH of 7.8 or a pH of 7.9;
   36—the GLP-1 compound is liraglutide, the polycationic compound is protamine, the liraglutide:protamine molar ratio is 1:0.13-0.15, the cores of the particles comprise no polycationic compound;
   37—the GLP-1 compound is liraglutide, the polycationic compound is protamine, the liraglutide:protamine molar ratio is 1:0.13-0.15, the core of the particles comprise no polycationic compound, in a composition having a pH≧7.7, a pH≧7.8, a pH between 7.7 and 8.0, a pH between 7.7 and 7.9, a pH of 7.7, a pH of 7.8 or a pH of 7.9;
   38—the GLP-1 compound is liraglutide, the polycationic compound is protamine, the liraglutide:protamine molar ratio is 1:0.11 or 1:>0.11, the cores of the particles comprise no polycationic compound, in a composition having a pH of 7.7 or a pH≧7.7;
   39—the GLP-1 compound is liraglutide, the divalent metal is zinc, the polycationic compound is protamine and the liraglutide:zinc:protamine molar ratio is 1:2.0-2, 4:0.13-0.15 or 1:2.1-2, 4:0.13-0.15;
   40—the GLP-1 compound is liraglutide, the divalent metal is zinc, the polycationic compound is protamine, the liraglutide:zinc:protamine molar ratio is 1:2.0-2, 4:0.13-0.15 or 1:2.1-2, 4:0.13-0.15, in a composition having a pH≧7.7, a pH≧7.8, a pH between 7.7 and 8.0, a pH between 7.7 and 7.9, a pH of 7.7, a pH of 7.8 or a pH of 7.9;
   41—the GLP-1 compound is liraglutide, the divalent metal is zinc, the polycationic compound is protamine, the liraglutide:zinc:protamine molar ratio is 1:2.0-2, 4:0.14-0.15 or 1:2.1-2, 4:0.13-0.15, and the cores of the particles comprise no polycationic compound;
   42—the GLP-1 compound is liraglutide, the divalent metal is zinc, the polycationic compound is protamine, the liraglutide:zinc:protamine molar ratio is 1:2.0-2, 4:0.14-0.15 or 1:2.1-2.4:0.13-0.15 the cores of the particles comprise no polycationic compound, in a composition having a pH≧7.7, a pH≧7.8, a pH between 7.7 and 8.0, a pH between 7.7 and 7.9, a pH of 7.7, a pH of 7.8 or a pH of 7.9;
   43—the GLP-1 compound is liraglutide, the divalent metal is zinc, the polycationic compound is protamine, the liraglutide:zinc:protamine molar ratio is 1:2, 2:0.13, 1:2, 2:0.14 or is 1:2, 2:0.15, in a composition having a pH≧7.7, a pH≧7.8, a pH between 7.7 and 8.0, a pH between 7.7 and 7.9, a pH of 7.7, a pH of 7.8 or a pH of 7.9;
   44—the GLP-1 compound and the divalent metal form an amorphous complex and/or a crystalline complex;
   47—any one of embodiments 27 to 44 wherein the particle size is less than 200 micrometers in diameter;
   48—any one of embodiments 27 to 44 wherein at least 50% of the particles have a volumetric diameter less than 60 μm;
   49—any one of embodiments 27 to 44 wherein at least 50% of the particles have a volumetric diameter less than 40 μm;
   50—any one of embodiments 27 to 44 wherein at least 50% of the particles have a volumetric diameter in the range of 5-35 μm;
   51—the GLP-1:divalent metal molar ratio in the core of the particle is 1:2 or 1:>2;
   52—the GLP-1:polycationic compound molar ratio in the particle is 1:>0,01,53—any one of the above embodiments, the particles being comprises in a pharmaceutical composition;
   54—a pharmaceutical composition comprising particles of any one of the above embodiments, the particles being suspended in an aqueous vehicle, in a non-aqueous vehicle, and/or incorporated into at least one biodegradable polymer.

In one aspect, the invention relates to a method of making particles as defined above or a composition comprising them.

The inventors found that GLP-1 compounds can be formulated into a sustained release suspension at rather high concentrations by carefully combining the active ingredient with two key components in a two-step approach: an initial mixing of the GLP-1 compound with a divalent metal salt, followed by an addition of a salt of a polycationic compound to the GLP-1:divalent metal mixture.

Therefore, in one embodiment, the method of the invention comprises one step of mixing of a GLP-1 compound with a divalent metal and one further step of adding a polycationic compound to the GLP-1 compound: metal mixture.

In one embodiment, the method of the invention comprises the following steps:

a) mixing of an aqueous solution of the divalent metal, the metal being in the form of a salt, with an aqueous solution of the GLP-1 compound, in order to form a suspension;
b) adding an aqueous solution of the polycationic compound, the polycationic compound being in the form of a salt, and an aqueous buffer solution to the suspension obtained from step a), the buffer being preferably added prior to adding the polycationic compound.

In one embodiment, the method also comprises a step c) wherein water is added to the mixture obtained from step b), in order to achieve the desired final concentration.

In one embodiment, for the purpose of step a), the GLP-1 compound is dissolved to prepare a stock at an appropriate concentration. The concentration of the GLP-1 stock solution can be in the range of 30-90 mg/mL, having a pH of about 8-9. A stock solution of the divalent metal is prepared with a concentration that can be in the range of 0.5-1.0 M range, having a pH of 5-7 depending of counter ion. In the case of zinc acetate, a 1 M solution has a pH value of about 6.6.

For the purpose of step a), the stock solutions can be mixed directly or pre-diluted before mixing. They are mixed under vigorous agitation or stirring using a magnetic stirrer or propeller in combination with an appropriate mixing container. Another option is to use a static mixer process, as known to persons skilled in the art. During this mixing step, the divalent metal and the GLP-1 compound co-precipitate, leading to an un-dissolved amorphous complex consisting of fine particles in the micrometer size range. It is a suspension of particles which form the core of the particles of the invention.

In one embodiment, in the mixing step a), the aqueous solution of GLP-1 compound is added sub-surfacially into the aqueous solution of metal salt, to avoid lump formation and/or the aqueous solution of GLP-1 compound has an alkaline pH and the aqueous solution of metal salt has an acidic pH. In one embodiment, the pH of the aqueous solution of GLP-1 is about 9.0. In one embodiment, the pH of the aqueous solution of metal salt is about 6.6.

In one embodiment, in the mixing step a), the metal salt solution is added to the GLP-1 solution. In another embodiment, in the mixing step a), the GLP-1 solution is added to the metal salt solution. This latter embodiment avoids the formation of small crystalline particles of zinc hydroxide, especially when the aqueous solution of GLP-1 compound has an alkaline pH and the aqueous solution of metal salt has an acidic pH.

In one embodiment, a buffer solution, such as a solution of TRIS, or TRIS containing a small amount of sodium hydroxide, is added after the mixing of step a) and before the addition of the polycationic compound, to achieve the pH of the final formulation. The use of sodium hydroxide alone, in a liraglutide solution, was not sufficient and the pH of the formulation was found to decrease by standing. The use of sodium phosphate, in a liraglutide solution, may lead to the formation of zinc phosphate crystals in the formulation during standing. For example, by addition of an unadjusted TRIS solution to a final formulation concentration of 25 mM prior to the addition of the polycationic compound, then only minimal or no final pH adjustment is necessary.

In one embodiment, for the purpose of step b), a stock solution of a salt of polycationic compound is prepared. The concentration of the stock solution can be at least 20 mg/mL, depending of the counterion of the chosen polycationic compound. The polycationic compound can be added directly from the stock solution or from a pre-dilution of the stock solution to the aqueous suspension containing the amorphous GLP-1:metal particles. This step results in a formulation comprising particles, the particles comprising a core, the core comprising or consisting of an amorphous and homogenous complex mixture of GLP-1 and divalent metal on which a layer of polycationic compound is layered in a complexed non-covalent manner.

In one embodiment, the stock solution of the polycationic compound is prepared with NaCl. It has been found that the solubility of the polycationic compound is significantly increased by adding NaCl, at relevant temperatures. This allows the storage and/or use of higher concentrations and lowers the volumes of polycationic compound stock solution needed. This makes much larger volume available for either the GLP-1 compound stock solution or the solution of metal salt. Thereby, it is easier to control the mixing process, involving the aqueous solution of GLP-1 compound and the stock solution of metal salt. As a result of this, it is easier to control the particle size of the core precipitate. The injectability of the final formulation is thereby improved, especially after storage at elevated temperatures. For example, a 30-60 mg/mL protamine sulphate stock solution is achievable at 21° C. when adding sodium chloride corresponding to a concentration of 0.3M, and a 30-80 mg/mL protamine sulphate stock solution is achievable at 21° C. when adding sodium chloride corresponding to a concentration of 0.5M.

The addition of a buffer solution, after the mixing of step a) and before the addition of the polycationic compound, as mentioned above, also allows the adjustment of the pH of the mixture of the above mentioned stock solutions. By carefully selecting the amount of buffer and sodium hydroxide, it is possible to avoid a final adjustment of the pH after addition of the polycationic compound. Thereby, an aseptic process is a lot easier to carry out successfully, by not having to take out an aliquot of the suspension in an aseptical manner, measure the pH and potentially adjust the pH by addition of sodium hydroxide and/or an appropriate acid until the target pH is reached.

In one embodiment, further excipients are added. For example, isotonic agents, such as glycerol or sodium chloride, are used to achieve isoosmolarity with blood serum. The isotonic agent does not need to be added as the last excipient. It can conveniently be added e.g. to the GLP-1 solution before addition to the metal salt solution.

Such particles may also be obtained be selecting an appropriate spray-drying process. The resulting GLP-1-divalent metal-polycationic compound containing particles may be resuspended into an aqueous or non-aqueous vehicle.

Therefore, in another embodiment, the method of the invention comprises the following steps:

a) preparation of a precipitate comprising the divalent metal and the GLP-1 compound;
b) spray-dry of the precipitate of step a) to form a powder;
c) re-suspension of the powder obtained from step b) in an aqueous medium or in a non-aqeuous medium;
d) addition of a solution of the salt of the polycationic compound and of a buffer to the suspension obtained from step c).

The following comments apply to the method above as well as to the methods below.

The preparation of step a) is operated like the mixing step a) described above.

For the purpose of step b), the spray-drying method is known to persons skilled in the art. An optimized particle size distribution is achieved.

For the purpose of step c), an aqueous medium can be a suitable iso-osmotic medium known by persons skilled in the art and a non-aqueous medium can be an pharmaceutical acceptable oil known to persons skilled in the art.

The buffer and polycationic compounds are added at step d) only for the purpose of obtaining an aqueous final formulation. In another embodiment, when a non-aqueous composition is desired, the addition of a solution of the salt of the polycationic compound and of a buffer of step d) is proceeded during the spray-dry of step b) rather than to the suspension obtained from step c). The addition of the polycationic compound during the spray-dry of step b) is also possible for the preparation of an aqueous final formulation. The buffer is added so as to achieve the pH desired for the final formulation. When added during the spray-dry of step b), the polycationic compound is added as a solution.

In one embodiment, the method is operated under aseptic conditions.

In another embodiment, the method of the invention comprises the following steps:

a) preparation of a precipitate comprising the divalent metal and the GLP-1 compound;
b) addition of a solution of a salt of the polycationic compound of a buffer to the precipitate of step a);
c) spray-dry of the precipitate of step b) to form a powder;
d) re-suspension of the powder obtained from step b) in an aqueous medium or in a non-aqueous medium.

In another embodiment, the method comprising the following steps:

a) preparation of a precipitate comprising the divalent metal and the GLP-1 compound;
b) high-pressure homogenisation and/or ultra-sound treatment of the precipitate of step a);
c) addition of an aqueous solution of a salt of the polycationic compound and of an aqueous buffer solution to the precipitate of step b).

For the purpose of step b), high pressure homogenizers (e.g an EmulsiFlex-05® obtainable from Avestin® Inc., Canada) and standard ultra-sound systems are known to people skilled in the art.

When the pharmaceutical composition is a non-aqueous suspension of particles, the particles are preferably obtained by isolation and drying from an aqueous suspension, or made by appropriate spray drying.

When the pharmaceutical composition is an aqueous suspension of particles, the particles are preferably used without isolation or made by appropriate spray drying.

The pharmacokinetic and pharmacodynamic properties of the resulting compositions can be evaluated by animal or clinical studies. The release properties of the compositions can also be evaluated by suitable in vitro release studies.

The chemical and physical stability of the resulting compositions can be evaluated by carrying out standard stability studies, making use of relevant analytical methods appropriate to characterize the GLP-1 compounds or selected excipients; and the composition as a whole.

In another aspect, the invention relates to particles and compositions obtained by the above described methods.

In one aspect, the invention relates to pharmaceutical compositions comprising particles as described above.

In another aspect, the invention relates to particles or pharmaceutical compositions comprising such particles, for use as a medicament.

In another aspect, the invention relates to particles or pharmaceutical compositions comprising such particles used for the treatment of metabolic diseases. Non-limiting examples of metabolic diseases include diabetes and obesity.

In another aspect, the invention relates to particles or pharmaceutical compositions comprising such particles, for use as a treatment by injection administration.

In another aspect, the invention relates to particles or pharmaceutical compositions comprising such particles, for use as a medicament with a frequency of administration below once per day (24 hours).

In one embodiment, the frequency of administration is below once per day and up to once per week (7 days), twice weekly or below twice weekly, 3 times weekly or below, 4 times weekly or below, 5 times weekly or below, or 6 times weekly or below.

In one embodiment, it is used for a treatment, especially the treatment of diabetes, by injection administration below once per day (24 hours) and up to once per week (7 days), twice weekly or below twice weekly, 3 times weekly or below, 4 times weekly or below, 5 times weekly or below, or 6 times weekly or below.

In one embodiment, it is used for a treatment, especially the treatment of diabetes, by injection administration once every 6 days, or once every 5 days, or once every 4 days, or once every 3 days, or once every 2 days, or less frequently than once every 1 day.

In one embodiment, it is used for a treatment, especially the treatment of diabetes, by injection administration once every 2 to 3 days, once every 3 to 4 days, once every 4 to 5 days, once every 5 to 6 days, once every 6 to 7 days, once every 5 to 7 days, once every 4 to 7 days, once every 3 to 7 days, or once every 2 to 7 days.

In one embodiment, the frequency of administration is below once weekly (7 days), below once monthly, or up to once per month (28, 29, 30 or 31 days), twice monthly or below twice monthly, 3 times monthly or below, 4 times monthly or below, or 5 times monthly or below.

In one embodiment, it is used for a treatment, especially the treatment of diabetes, by injection administration below once weekly (7 days), below once monthly, or up to once per month (28, 29, 30 or 31 days), twice monthly or below twice monthly, 3 times monthly or below, 4 times monthly or below, or 5 times monthly or below.

In one embodiment, it is used for a treatment, especially the treatment of diabetes, by injection administration once every 27-31 days, or once every 22-27 days, or once every 19-21 days, or once every 15-20 days, or once every 12-15 days, or once every 8-12 days, or less frequently than once every week.

In one embodiment, it is used for a treatment, especially the treatment of diabetes, by injection administration once every 27-31 days, or once every 22-27 days, or once every 19-21 days, or once every 15-20 days, or once every 12-15 days, or once every 8-12 days, or less frequently than once every week.

### EXAMPLES

(a) Morphology of the Particles

The specific morphology of the particle of the invention, i.e. a zinc-liraglutide core and protamine covering the surface of this core, has been demonstrated by various analytical techniques (data not shown):

i. Polarised microscopy analysis show that the final suspension is of amorphous nature.

ii. Confocal Raman spectroscopy data support the hypothesis that protamine predominantly is present at or near the surface of liraglutide-zinc particles. Furthermore, FTIR (Fourier transform infrared) spectroscopy data support that liraglutide is kept intact within the particles, and in a heptameric form. In addition, that liraglutide and zinc is present in the core, in a homogenous manner.

iii. Upon treating the final particle suspension with a 40 mM hydrochloric acid solution it all dissolves. By treating the final suspension with a 3.2 mM hydrochloric acid solution liraglutide-zinc particles are still present and all the protamine in the final particle suspension is dissolved into the 3.2 mM hydrochloric acid solution.

(b) Liraglutide-Zinc-Protamine Particles Compared to Compositions with No Protamine or with No Zinc

It has been found that preparing a mixture of aqueous liraglutide and zinc salt solutions results in a suspension of amorphous particles consisting of liraglutide:zinc. Upon subcutaneous injection to pigs, this suspension leads to a PK profile with no significant sustained release plasma profile, as compared to a solution of liraglutide.

It has also been found that mixing liraglutide with protamine does not result in a suitable product or suspension. Only a very sticky mixture with no particle size control is obtained.

However, the following setup has been found to work out well: a combination of liraglutide with zinc ensures a suspension of particles of appropriate size. Upon subsequent addition of protamine to the liraglutide-zinc particles suspension, the liraglutide-zinc particles are covered with a sufficient layer of protamine. The obtained suspension is resuspendable upon long term storage at 5° C. or upon short term storage at 25° C. or more. It is injectable through fine injection needles, such as 30 G (gauge) TW (Thin Walled) needles, both into air and into subcutaneous tissue. The resulting product gives a suitable sustained release PK profile upon subcutaneous administration.

(c) A Pharmaceutical Composition of the Invention:

In one embodiment, the pharmaceutical composition of the invention comprises:

| Concentration (mM) | Item | Concentration (mg/mL) |
|---|---|---|
| 10.7 or 10-11.5 | Liraglutide | 40 mg/ml |
| 23.5 or 22.5-24.5 | Zinc Acetate | 4.3 mg/ml |
| 1.5 or 1.4-1.6 | Protamine Sulphate | 8.0 mg/ml |
| 25 or 24-26 | Tris | 3.0 mg/mL |
| 72 or 70-74 | Sodium Chloride | 4.2 mg/ml |

pH 7.8
Here the molar ratio of liraglutide:zinc:Protamine is [1:2.2; 0.14]

This pharmaceutical composition presents the following beneficial properties:

a sustained release PK-plasma profile, in pig model, with minimal burst release of liraglutide;

a minimum of histamine release, in rat model;

an acceptable low tissue reaction, as shown by histology study in pigs;

a good chemical stability;

an acceptable physical suspension stability;

no liraglutide in supernatant: much less than 0.1% of the total liraglutide concentration;

a minimum zinc and protamine in supernatant;

injectable through a 30 G TW needle into either air or subcutaneous tissue at an acceptable dosing rate.

In another embodiment, the pharmaceutical composition of the invention comprises:

| | | |
|---|---|---|
| Liraglutide | 10-11.5 | mM |
| Zinc Acetate | 22.5-24.5 | mM |
| Protamine Sulphate | 1.4-1.6 | mM |
| Tris | 24-26 | mM |
| Sodium Chloride | 70-74 | mM |
| pH | 7.7-7.9 | mM |

(d) A method of the Invention:

2.35 ml of a 0.5 M Zn(OAc)₂ solution and 5.0 ml of a 0.3 M NaCl solution are added to a 100 ml beaker containing a magnetic stirrer pin. 25 ml of an aqueous solution of 80 mg/ml liraglutide is added with heavy agitation by a thin cannula beneath the surface. After agitation for 5 minutes, 625 μl of a 2 M TRIS solution (tris(Hydroxymethyl)ami-nomethane) and 100 μl of a 1M NaOH are added. After further agitation for 10 minutes, 8.4 ml of a 50 mg/ml solution of Protamine sulphate in 0.3 M NaCl is added. Water is finally added q.s. (in sufficient quantity) to a formulation volume of 50 ml. The liraglutide:zinc:protamine final molar ratio is 1:2, 2:0.15.

The pH of the final formulation is 7.7-7.9

(e) A Method of the Invention:

2.26 gram of liraglutide (88.4% protein) is dissolved at 23° C. in 22 ml of water and 1.20 ml 3 M NaCl is added and the solution is sterilised by filtration. 1175 μl 1 M Zinc acetate is mixed with 1.0 ml water, sterilised by filtration and added to a preweighed 100 ml sterile borosilicate bottle (BlueCap, Duran®) equipped with a magnet stirrer pin. The liraglutide solution is added under vigorous agitation (about 400 rpm) to the zinc solution beneath the surface at the bottle wall through a long 23 G cannula as fast as possible. After further 2 minutes agitation 1250 μl sterilised 1 M TRIS solution is added and followed by 20 ml of sterilised 2% protamine sulphate solution. Sterile water is added up to a formulation weight of 50 gram and the suspension is slowly agitated for 1 hour. The pH is finally adjusted to 7.8 with 2 M NaOH. The bottle is closed and left overnight at 5° C. Next morning the pH is checked at 23° C. and readjusted if necessary to 7.8. The suspension is then filled into injection pens (Penfill® cartridges). The liraglutide:zinc:protamine final molar ratio is 1:2, 2:0.14.

(f) Zinc Mediated Precipitation of Liraglutide at Various pH-Values:

Liraglutide has been found to precipitate quantitatively from a solution containing Zinc ions. FIG. 1 shows the concentration of free liraglutide (y-axis), in mg of liraglutide per mL of supernatant, for increasing values of zinc:liraglutide molar ratio x-axis) and various pH. A low concentration of free liraglutide indicates a high rate of liraglutide co-precipitation with zinc, and inversely.

In this test, stock solutions of liraglutide were made in 12.5 mM tris buffer and contained 50 mM liraglutide. A zinc acetate stock solution (213.2 mM) was prepared.

For each sample, an appropriate amount of zinc acetate stock solution was added to 0.4 mL of liraglutide stock solution. MilliQ was added to each sample to reach a final volume of 0.5 mL. After whirli-mixing pH was adjusted to 7.8, 8.0, or 8.2. After pH adjustment the samples where whirli-mixed once more. After sedimentation of the particles, supernatant was withdrawn, centrifuged (15.000 G for 15 minutes) and the content of liraglutide in the supernatant was analyzed by UV-spectroscopy.

The results, reported on FIG. 1, show that the rate of liraglutide precipitation depends on the zinc:liraglutide molar ratio. At pH 7.8, a minimum molar ratio of 2.0-2.2:1, or 2, 1:1 is required to obtain no free liraglutide in the supernatant, i.e. 100% of liraglutide is precipitated. At pH 8.0, the minimum molar ratio is 2.1-2, 2:1, and at pH 8.2, the minimum molar ratio is >3:1. At pH 7.0, the minimum molar ratio of zinc to obtain 100% liraglutide precipitated is 1.3 (data not reported).

(g) Protamine Binding to Precipitated Zinc-Liraglutide Complex:

With a zinc:liraglutide molar ratio of 2, 2:1 as set point, the molar ratio of protamine bound to zinc-precipitated liraglutide was investigated.

In this test, a suspension of co-precipitated zinc and liraglutide was prepared so as to comprise 10 mM of liraglutide, 22 mM of ZnCl₂ (in the form of Zn2+ in the co-precipitate), which means that the zinc:liraglutide molar ratio is 2, 2:1. The pH of the final suspension was 7.8, at 25° C.

Therefore, an amount of liraglutide bulk material corresponding to 1,875 gram of liraglutide (corresponding to 0.5 moles) was dissolved in 30 mL of water. After filtration, 5.5 ml of 0.2 M ZnCl₂ was added to the filtrate containing liraglutide, under vigorous stirring. Afterwards, 1 ml of 1 M Tris buffer, (pH 7.8) was added. Hereafter, the pH was adjusted from pH 7.5 to 7.8 by addition of about 10 μl of 1M NaOH. In each of 8 containers, 10% of the resulting suspension was transferred. Under vigorous stirring, a specific portion of a 18 mM Protamine Chloride solution was added.

The initial container received 0.10 mL of the 18 mM Protamine Chloride solution, followed by 0.2 mL to the second container; ending with addition of 0.8 mL to the final container (number 8). Water was added to each of the eight containers to achieve a total of 5.0 grams of suspension. After 1 hour storage, about 1 mL were withdrawn from each container and centrifuged for 10 minutes at high speed. A portion of clear supernatant was withdrawn from each container and the amount of free Liraglutide and protamine. was measured by a standard liquid chromatographic method.

FIG. 2 shows the concentration of free protamine (y-axis left) and the concentration of free liraglutide (y-axis right) in solution (i.e. the supernatant), in mM, for increasing concentrations of protamine x-axis), in mM, added to the particles, at pH 7.8 and 25° C. A low concentration of free liraglutide indicates a high rate of liraglutide present in the particles, a low concentration of free protamine indicates a high rate of protamine present on the particles, and inversely.

The results, reported on FIG. 2, show that the maximal amount of protamine bound to the suspensed particles is 0.1 mol Protamine per mol liraglutide. The amount of protamine added to the suspension does not influence the solubilisation of free liraglutide.

To ensure a sufficient sustained release of liraglutide, a slight surplus of protamine is convenient. Therefore a ratio of 0.14 or 0.15 mol protamine to 1 mol liraglutide is selected.

Liraglutide:zinc:protamine molar ratios of 1:2, 1:0.13 or 1:2, 2:0.13, 1:2, 1:0.14 or 1:2, 2:0.14 or 1:2, 1:0.15 or 1:2, 2:0.15 are selected, with pH 7, 8 or 7.7-7.9.

(h) Liraglutide and Protamine in Solution at Different pH-Values in a Suspension Formulation with a Molar Liraglutide:Zinc:Protamine Ratio of 1:2, 2:0.14:

The binding of protamine to the zinc-precipitated liraglutide is dependant of the pH value of the formulation. Here, the amount of protamine remaining in solution was determined in formulations with different pH-values.

In this test, a suspension of co-precipitated zinc and liraglutide was prepared so as to comprise 10.7 mM of liraglutide, 23.5 mM of ZnCl₂ (in the form of Zn2+ in the co-precipitate), 1.5 mM protamine sulphate, 40 mM TRIS/HCl, 60 mM NaCl, so that the liraglutide:zinc:protamine molar ratio is 1:2, 2:0.14. The pH of the final suspension was tested within the range 7-8, at 23° C., by appropriate adjustment of the final pH. The other details on this experiment is similar to the method in example (g). For each tested pH, both free liraglutide and free protamine were measured in the supernatant by a standard liquid chromatographic method.

FIG. 3 shows the concentration of free protamine (y-axis left) and the concentration of free liraglutide (y-axis right) in solution (i.e. the supernatant), in mM, for increasing pH values. A low concentration of free liraglutide indicates a high rate of liraglutide present in the particles, a low concentration of free protamine indicates a high rate of protamine present on the particles, and inversely.

The results, reported on FIG. 3, show that the minimal protamine concentration in the supernatant was found at pH 7.7-7.9. Thus a pH-value of 7.7-7.9 or of 7.8 is selected.

(i) Liraglutide Concentration:

Pharmacokinetic studies (data not reported) have shown that a formulation according to the invention with a liraglutide concentration of 40 mg/ml (10.7 mM) is sufficient for achieving an acceptable level of liraglutide in the bloodstream lasting for 7 days.

(j) Formation of the Liraglutide-Zinc-Protamine Suspension:

Liraglutide can be precipitated just by convenient pH adjustment (isoelectric precipitation) or by addition of zinc ions and/or of a protamine salt. Precipitation of liraglutide from the highly concentrated stock solution by pH adjustment alone results in serious gelation of the formulation and this is also the case when precipitated by protamine salt addition alone or by simultaneous addition of protamine salt and zinc salt.

By precipitation with zinc salt alone a readily resuspendable precipitation with a suitably particle size distribution can be achieved. By addition of the protamine after the zinc-precipitation these properties of the particles can be maintained and the protamine binds to the outer parts of the particles and form a surface cover that will further delay the dissolution of the liraglutide particles.

(k) Suitable Excipient Stock Solutions for the Manufacturing Process of a Liraglutide-Zinc-Protamine Suspension:

| | |
|---|---|
| 90 mg/ml Liraglutide | 1M Zinc Acetate |
| 1M tris(hydroxymethyl)aminomethane (TRIS | 20 mg/ml Protamine |
| | Sulphate |
| 3M NaCl (Isotonic agent) | 2N NaOH (pH adjustment) |

These solutions are sterile and are applicable for manufacturing of a once weekly liraglutide suspension of the formulation given in example (c).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A particle comprising a GLP-1 compound, a divalent metal and a polycationic compound, wherein said particle comprises a core and a surrounding layer, the core comprising the GLP-1 compound and the divalent metal, and the surrounding layer comprising the polycationic compound.

2. The particle according to claim 1, wherein the GLP-1 compound comprises a natural GLP-1, an analogue of GLP-1, a derivative of GLP-1, liraglutide, semaglutide, taspoglutide, exenatide, lixisenatide, albiglutide, dulaglutide or N ³⁷-[2-[2-[2-[[2-[2-[2-[[(4S)-4-carboxy-4-[[4-[(19-carboxynonadecanoylamino)methyl]cyclo hexanecarbonyl]amino]butanoyl]amino]ethoxy]ethoxy]acetyl]amino]ethoxy]ethoxy]acetyl]-[Imp⁷,Glu²²,Arg²⁶,Arg³⁴,Lys³⁷]-GLP-1-(7-37)-peptide or N ²⁶-[2-[2-[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl], N ³⁷-[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide or N ²⁶-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl],N ³⁷-[2-[2-[2-[[2-[2-[2-[[(4S)-4-carboxy-4-[12-(3-carboxyphenoxy)dodecanoylamino]butanoyl]amino]ethoxy]ethoxy]acetyl]amino]ethoxy]ethoxy]acetyl]-[Aib⁸,Arg³⁴,Lys³⁷]-GLP-1-(7-37)-peptide.

3. The particle according to claim 1, wherein the divalent metal is selected from zinc (Zn), calcium (Ca), manganese (Mn) or magnesium (Mg).

4. The particle according to claim 1, wherein the polycationic compound is selected from protamine, chitosan, a chitosan derivative, polylysine and polyarginine.

5. The particle according to claim 1, wherein the GLP-1 compound and the divalent metal form a homogenous mixture.

6. The particle according to claim 1, wherein the core comprises substantially no protamine.

7. The particle according to claim 1 with a pH between 4 and 8.2 when dissolved or suspended in water, or comprised in a composition having a pH between 4 and 8.2.

8. A pharmaceutical composition comprising particles according to claim 1.

9. A method for the preparation of a particle as defined in claim 1, comprising one step of mixing of a solution of a GLP-1 compound with a solution of a divalent metal and one further step of adding a solution of a polycationic compound to the GLP-1 compound: metal mixture.

10. The method of claim 9, comprising the following steps:
a) mixing of an aqueous solution of the divalent metal, the metal being in the form of a salt, with an aqueous solution of the GLP-1 compound, in order to form a suspension;
b) adding an aqueous solution of the polycationic compound, the polycationic compound being in the form of a salt, and an aqueous buffer solution to the suspension obtained from step a), the buffer being preferably added prior to the polycationic compound;
c) adding water to the mixture obtained from step b).

11. The method of claim 10 wherein, in the mixing step a), the aqueous solution of GLP-1 compound is added sub-surfacially into the aqueous solution of metal salt, and/or wherein the aqueous solution of GLP-1 compound has an alkaline pH and the aqueous solution of metal salt has an acidic pH.

12. The particle as defined in claim 1 for use as a medicament.

13. The particle as defined in claim 1 for use as a medicament in the treatment of metabolic diseases.

14. The particle as defined in claim 1 for use as a medicament with a frequency of administration below once per day.

15. The pharmaceutical composition as defined in claim 8 for use as a medicament.

16. The pharmaceutical composition as defined in claim 8 for use as a medicament in the treatment of metabolic diseases.

17. The pharmaceutical composition as defined in claim 8 for use as a medicament with a frequency of administration below once per day.
